(19) United States
(12) Patent Application Publication
(10) Pub. No.: US 2013/0260400 A1
(43) Pub. Date: Oct. 3, 2013

(54) ASSAY FOR A TYPE II COLLAGEN BIOMARKER

(71) Applicants: Anne-Christine B. Jensen, Copenhagen S (DK); Qi Liu, Beijing (CN); Jianxia Wang, Beijing (CN)

(72) Inventors: Anne-Christine B. Jensen, Copenhagen S (DK); Qi Liu, Beijing (CN); Jianxia Wang, Beijing (CN)

(73) Assignee: Nordic Bioscience A/S, Herlev (DK)

(21) Appl. No.: 13/825,795

(22) PCT Filed: Sep. 16, 2011
(86) PCT No.: PCT/EP11/66076

§ 371 Date: Jun. 13, 2013

(30) Foreign Application Priority Data
Sep. 24, 2010 (GB) 1016050.5

(51) Int. Cl. G01N 33/68

(52) U.S. Cl. CPC G01N 33/6887
USPC 435/7.92; 435/287.2

(57) ABSTRACT

An assay for Type II collagen fragments in serum, plasma, or synovial fluid obtains a quantitative measure of the concentration of all protein fragments in a serum, plasma, or synovial fluid sample that are reactive with an antibody, or immunoreactive antibody fragment, having specific reactivity with a C-terminal epitope present in the amino acid sequence GPPGRDGAAG and lacking specific reactivity with an amino acid sequence comprising the amino acid sequence GPPGRDGAAGV, which may be Mab NB44-3C1 as produced by the cell line deposited in HPA Culture Collection Logistics Office with Accession Number 10091402.

The present invention relates to measurement in serum and other body fluids of fragments of Type II collagen having significance as a measure of cartilage destruction in osteoarthritis (OA) and/or rheumatoid arthritis (RA).

Type II collagen is the primary matrix protein in articular cartilage. In joint degenerative diseases, such as osteoarthritis (OA) and rheumatoid arthritis (RA), the collagens are degraded by matrix metalloproteinases (MMPs) and fragments of the protein are released into the circulation.

In WO2010/055062 we described an MMP generated cleavage product of Type II collagen giving rise to a C-terminal epitope in a sequence GSPGADGPPGRDGAAG_{↓}. We proposed sandwich assays measuring fragments containing an isomerised amino acid residue and a protease generated neoepitope such as this.

In WO2010/055064 we described assays for biomarker fragments combining two MMP generated neo-epitopes from Type II collagen and proposed a sandwich assay directed to the sequence LTGPAGEPGREGSPGADGPPGRDGAAG with antibodies directed to epitopes LTGPAG . . . and . . . RDGAAG. Measurements were made of reactive fragments released from human cartilage under anabolic conditions of culture and under catabolic conditions of culture. Measurements were also made in human urine samples from OA and control patients, showing an increase of the relevant fragments in OA.

We have now found that good results are obtainable from an assay for Type II collagen fragments in serum reactive with a monoclonal antibody raised against the C-terminal sequence . . . RDGAAG without the need for the use of a second antibody.

As disclosed in WO2010/055064 we located MMP cleavage sites in Type II collagen by the following procedure. Human collagen type II (BIOCOL BC-3001) was dissolved in 10 mM acetic acid (400 μl added to 1 mg of collagen type II). Ten pg of procathepsin K (Calbiochem 342001) was activated by addition of 200 μl of 100 mM sodium acetate containing 10 mM DTT and 5 mM EDTA, pH 3.9 for 40 minutes at room temperature. Ten pg of MMP9 (Calbiochem 444231) was activated by addition of 200 μl of 1 mM APMA in DMSO for 2 hours at 37° C. For the Cathepsin K cleavage, 60 μl of collagen type II was added 120 μl of 50 mM sodium acetate, pH 5.5 containing 20 mM L-cystein and 24 μl of activated cathepsin K for 4 hours at 37° C. For the MMP9 cleavage, 60 μl of collagen type II was added 120 μl of 100 mM Tris-HCl, 100 MM sodium chloride, 10 mM calcium chloride, 2 mM zinc chloride, pH 8.0 and 20 μl of MMP9 for 3 days at 37° C. The resulting proteolytic cleavage fragments were characterized by high performance liquid chromatography (HPLC)-tandem mass spectrometry (MS/MS) analysis. The MS/MS spectra were searched against protein databases using Sequest and X! Tandem database search algorithms.

Cleavage sites were localized as indicated in the following annotated sequence of Type II collagen, where Cathepsin K-generated sites are marked by arrows and MMP9-generated sites by stars.

QMAGGFDEKAGGAQL*GVM*QGPMGPM*GPRGPPGPAGAPGPQG*FQGNPGEPGEPGVSGP
MGPR [symbol] GPPGPPGKPGDDGEAGKPG [symbol] KAGERGPPGPQGARGFPGTPGLPGVKGHRGYPGLDG
AKGEAGAPG*VK* [symbol] GESGSPGENG*SPGPM* [symbol] GPRG*LPGERGRTGPAG*AAGARGNDGQP
GPA [symbol] GPPGPV [symbol] GPA* [symbol] GGPGFPGAPGAKGEAGPTGARGPEG*AQGPRGEPGTPGSPGPAG*
ASGNPGTDGIPGAKGSAGAPGIAGAPGFPGPRGPPGPQG*ATGPLGPKG*QTGEPGIAGFK
GEQGPKGEPGPAGPQGAPGPAGEEGKRGARGEPGGVG [symbol] PIGPP [symbol] G*ERGAPGNRGFPGQDG
LAGPKGAPGERGPSG*LAGPKGANGDPGRPGEPGLPGARG*LTGRPGDAGPQG*KVGPS* [symbol]
GAPGEDGRPGPPGPQ* [symbol] G*ARGQPGVMGFPGPKGANGEPGKAGEKGLPGAPGLRG*LPGKD
GETGAAGPPGPAGPAG*ERGEQGAPGPSG*FQ [symbol] G [symbol] LPGPPGPPGEGGKPG [symbol] DQGVPGEAGA
PGLVGPR* [symbol] G*ERGFPG [symbol] ER [symbol] GSPGAQGL*QGPRGLPGTPGTDGPKGASGPAGPPGAQGPP
*GLQGMPGERGAAGIAGPKGDRGDVGEKGPEGAPGKDGGRG*LT* [symbol] GPIGPPGPA* [symbol] G*AN
GEKGEVGPPGPA* [symbol] GSA [symbol] G*AR*GAPGERGETGPPGPA* [symbol] G*FAGPPGADGQPGAKGEQGE
AGQKGDAGAPGPQGPSGAPGPQGPTGVTGPKGARG [symbol] AQGPPGATGFPGAAG*R [symbol] VGPPG* [symbol]
SNGNPGPPGPPGPS* [symbol] G*KDGPKGARGDSGPPGRAGEPGLQGPAGPPGEKGEPGDDGPSGA
EGPPGPQGLAGQRG*IVGLPGQRGERGFPGLPGPSGEPGK*QGAPG* [symbol] AS [symbol] G*DR*GPPGP
V* [symbol] G*PPG* [symbol] LTGPAGEPG [symbol] REGSPGAD*GPPGRDGAAG*VK [symbol] GDRGETGAVGAPGAPG
PPGSPGPAGPTG*KQGDRGEAGAQGPMGPSGPAG*ARGIQGPQGPRGDKGEAGEPGERGLK
GHRG*FTG*LQGLPGPPGPSG*DQGASGPAGPSGPRGPPGPVGPSG*KDGANGIPGPIGPP
GPRG*RSGETGPAGPPG [symbol] NPGPPGPPGPPGPG [symbol] ID*MSAFAGLGPREKGPDPLQYMRA

In particular, we identified an MMP cleavage fragment (CIIM) of MMP-derived type II collagen, i.e. the neoepitope identified as RDGAAG¹⁰⁵³ having a free C-terminal Glycine residue. This fragment is marked in bold. Further we have developed a particularly advantageous monoclonal antibody (NB44-3C1) having specific reactivity with this neoepitope sequence. The Applicant has made the cell line expressing this monoclonal antibody the subject of a Budapest Treaty deposit with the HPA Culture Collection Logistics Office, Health Protection Agency Culture Collections, Centre For Emergency Preparedness and Response, Porton Down, Salisbury Wiltshire, SP4 0JG, UK, with Accession Number 10091402.

The currently available information available to the Applicant regarding the characteristics of the cell line is as follows. The cell line secretes a monoclonal antibody we designate NB44-3C1.

Presently, evidence is available to demonstrate a strong reactivity of monoclonal antibody NB44-3C1 to a peptide having the amino acid sequence PPGRDGAAG originating from type II collagen (as indicated above), while having only little (if any) reactivity to the elongated peptide having the amino acid sequence PPGRDGAAGV, i.e. elongated with one amino acid in the C-terminus. No binding of monoclonal antibody NB44-3C1 could be detected to a range of peptides fragments not encompassing the amino acid sequence PPGRDGAAG, and these non-reactive peptides included ETGERGAAG, GFGLPGAAG, DQGVPGEAG, REGSPGASG, LAGPKGANG, DDGEAGKPG, ATGPLGPKG, LTGPAGEPG, and GADGPPGRD. Furthermore, monoclonal antibody NB44-3C1 binds strongly to collagen type II pretreated with matrix metalloproteinases, whereas the reactivity to type II collagen not pretreated with MMPs was less than 3%. No binding to type III collagen with or without pretreatment with MMPs could be detected. Finally, monoclonal antibody NB44-3C1 binds strongly to antigens present in human serum, plasma and synovial fluid. Surprisingly it was found that monoclonal antibody NB44-3C1 recognising the neo-epitope RDGAAG¹⁰⁵³ bound to antigens in human circulation, which upon quantification provided superior clinical utility, e.g. capability to distinguish groups of patients with joint disease from healthy individuals.

Accordingly, the present invention provides a method of assay for Type II collagen fragments in serum, plasma, or synovial fluid comprising obtaining a quantitative measure of the concentration of protein fragments in a serum, plasma, or synovial fluid sample that are reactive with an antibody, or immunoreactive antibody fragment, having specific reactivity with a C-terminal epitope present in the amino acid sequence GPPGRDGAAG and lacking specific reactivity with an amino acid sequence comprising the amino acid sequence GPPGRDGAAGV.

Preferably, no subset of protein fragments that are reactive with said antibody, or immunoreactive antibody fragment, having specific reactivity with a C-terminal epitope present in the amino acid sequence GPPGRDGAAG and lacking specific reactivity with an amino acid sequence comprising the amino acid sequence GPPGRDGAAGV are excluded from the measurement. This requirement is not consistent for instance with the use of a further antibody in a sandwich format, as in such a format fragments having the C-terminal sequence RDGAAG but lacking any sequence reactive with the second antibody would be excluded from the measurement.

More specifically therefore, the assay is preferably not conducted in a sandwich format.

Preferably the method comprises contacting protein fragments present in a said serum sample with a said antibody or immunoreactive antibody fragment and measuring the amount of binding of said fragments thereto.

The serum, plasma, or synovial fluid sample may be subjected to some processing prior to analysis if desired, provided that relevant protein fragments are still present for measurement.

Preferably, said antibody or immunoreactive antibody fragment is contacted with both said protein fragments and a competition agent for which said antibody or immunoreactive antibody fragment has specific binding affinity.

Preferably, the antibody is NB44-3C1 as deposited in HPA Culture Collection Logistics Office, Health Protection Agency Culture Collections, Centre For Emergency Preparedness and Response, Porton Down, Salisbury Wiltshire, SP4 0JG, UK, with Accession Number 10091402.

The invention includes a said antibody, which may be labelled with a detectable label such as a peroxidase enzyme label, e.g. horse radish peroxidase.

The invention further includes a test kit for conducting an immunoassay comprising antibody NB44-3C1 as described above, together with one or more of, standards for generating a calibration curve, a peptide immunologically reactive with said antibody, a chromogenic substrate capable of participating in a colour forming reaction (for instance TMB), a stopping solution for a colour forming reaction, and a multi-well assay plate.

The assay kit is preferably for the conduct of an immunoassay using the antibody in a competition format, which may be ELISA or RIA.

In particular, the peptide which is reactive with the antibody, and which serves as a competition agent, may be bound to a solid surface, e.g. may be conjugated to biotin and bound to a streptavidin coated multi-well plate.

In an alternative format, the antibody may be bound to a solid surface, e.g. may be conjugated to biotin and may be bound to a streptavidin coated multi-well plate.

The invention will be further described and illustrated by the following Examples, making reference to the accompanying drawing in which:

FIG. 1 shows in panels A to F immunostaining of human cartilage to show localisation of the CIIM fragment in OA cartilage;

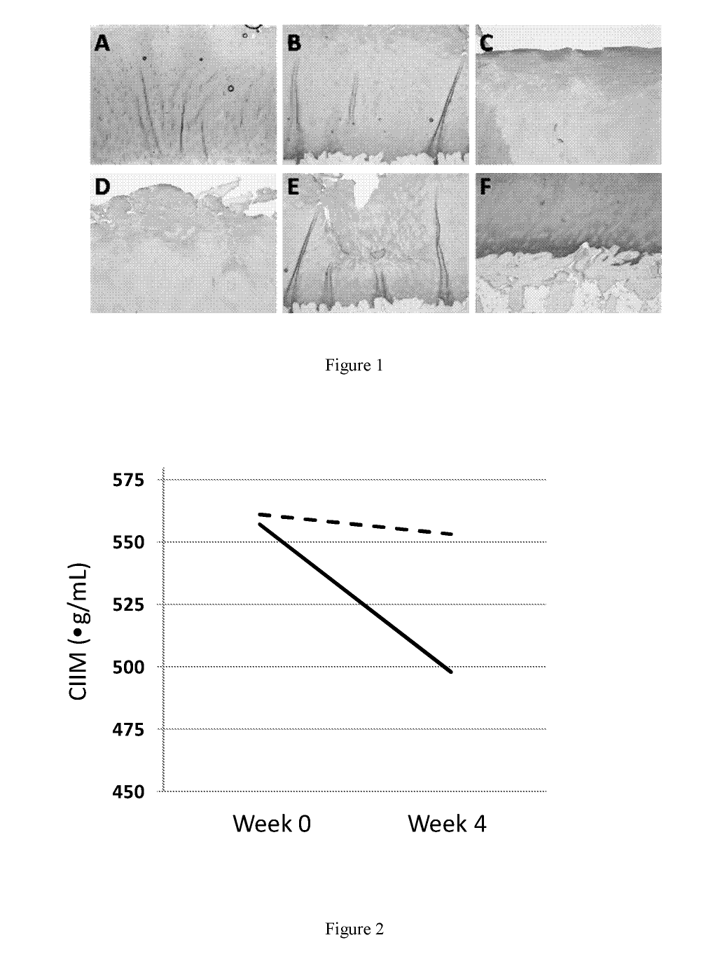

FIG. 2 shows results obtained in Example 4 measuring the CIIM fragment in human serum;

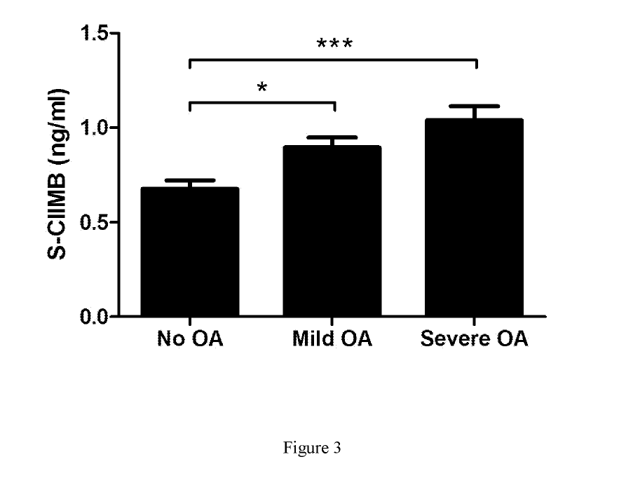

FIG. 3 shows serum levels of CIIM reactivity correlated with KL scores (Example 5); and

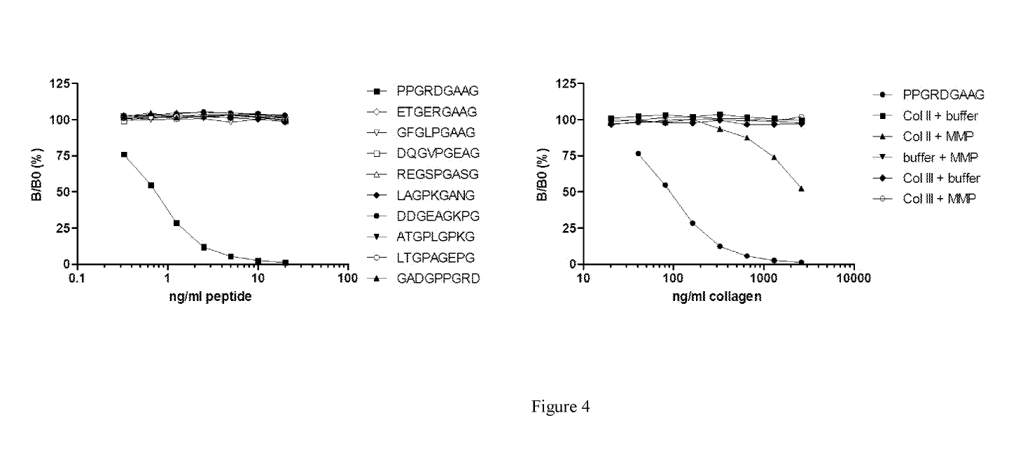

FIG. 4 shows comparative reactivity of the antibody of the invention with various peptides.

### EXAMPLE 1.

### Development of the Antibody NB44-3C1

The immunogenic peptide, C-GGGRDGAAG, was conjugated to keyhole limpet hemocyanin (KLH) according to standard operational procedures. Briefly, 5 mg/ml of the cysteine-containing peptide, C-GGGRDGAAG, and the maleimide-activated KLH, were mixed in conjugation buffer and incubated for 2 hours at room temperature. The conjugated antigen was purified by desalting and dialysis to remove EDTA and sodium azide.

Six 8-weeks old Balb/C mice were immunized subcutaneously with 200 μL emulsion containing equal volumes of Freund's incomplete adjuvant and of immunogen, KLH-C-GGGRDGAAG. Consecutive immunizations in Freund's incomplete adjuvant were performed with 30 μg/mouse immunogen every second weeks for 3 immunizations, and subsequently every 4^{th} week. The mice were bled from the 2^{nd} immunization onwards. At each bleeding, the serum titers were detected against selection peptide (PPGRDGAAG), de-selection peptide (PPGRDGAAGV), and native material, in-house sample array (data not shown) on streptavidin plates coated with biotinylated selection (Biotin-KPPGRDGAAG) or deselection peptide (Biotin-KPPGRDGAAGV). Two mice, with the highest serum titers and good native reactivity, were selected for fusion. The selected mice were rested for 1 month before they were boosted intravenously with 50 μg immunogen in 100 μL 0.9% sodium chloride solution. Three days later, the mice were terminated and the spleen cells were isolated. Fusions (hybridomas) were performed with SP2/0 cell (LGC Standards AB, Boras, Sweden), according to standard procedures.

The hybridomas were cloned in 35 mm cell culture Petri-dishes using the semi-solid medium method. Different hybridomas picked up from the dishes individual were transferred to wells of 96-well plates where the cells were cloned by limited dilution. Clones were screened in ELISA. Briefly, the 10 ng/ml biotin-labeled selection and deselection peptides were coated onto streptavidin plates for 30 mins at 20° C. in 10 mM PBS-BTB (10 mM PBS including BSA, Tween-20 and bronidox). After being washed 5 times in standard washing buffer, 20 μl test material (i.e. displacement peptide or native material) was added, to the appropriate wells. Without washing, 100 μl clone supernatant in a 2-fold dilution starting at 1:100 was added and incubated for 2 hours at 20° C. The wells were then washed and a peroxidase-labelled secondary antibody against mice IgG was added [1:3000] (Jackson ImmunoResearch, Europe Ltd, UK) and incubated for 1 hour at 20° C. After washing, 100 μl of tetramethylbenzidine was added (TMB (3,3′,5,5”-tetramethylbenzidine), Kem-En-Tec, Roedovre, Denmark) followed 15 minutes later by 100 μl stop solution (0.18 M sulfuric acid). The colorimetric assay was measured at 450 nm using 605 nm as reference. Selection peptides were used to confirm the binding of the antibodies to the target sequence, whereas the deselection peptides were used to determine any reactivity of the antibodies to fragments not carrying the target sequence. Selected clones were then subcloned twice and monoclonal antibodies purified using Protein G columns according to manufacturer's instructions.

Through two subclonings, cell line NB44-3C1 could repeatedly be demonstrated to have the desired reactivity profile using the selection and deselection peptides, strong reactivity to fragments in human serum, plasma and synovial fluid, and finally had a high degree of viability and antibody-production capacity.

### EXAMPLE 2

### Conduct of a Competition CIIM ElISA

The CIIM competitive ELISA procedure was as follows: a 96-well streptavidin-coated plate was coated with 4 ng/ml Biotin-KPPGRDGAAG for 30 min, at 20° C., at 300 rpm. The wells were then washed 5 times with standard washing buffer. A standard row was prepared by pre-dilution [2.5-fold] of the specific peptide (PPGRDGAAG) in 10 mM PBS-BTB. Samples (i.e. peptides, human serum, plasma or synovial fluid) were likewise diluted. Standards and samples were added [20 μl/well], followed by the addition of 100 μl/well of 20 ng/ml peroxidase-labeled NB44-3C1 antibody. The wells were then covered and incubated overnight (18±1 hours) at 4° C., and at 300 rpm. Then the wells were washed 5 times and incubated with 100 μl of TMB at 20° C., 300 rpm, for 15 min, followed by the addition of 100 μl stop solution of 100 μl/well in each well. The colorimetric reaction was measured at 450 nm with reference at 650 nm on a standard laboratory plate reader. Data was acquired with the SoftMax Pro v5.0 program.

The CIIM ELISA showed good technical performance; a quantification range of 0.2-14.9 ng/ml, inter- and intra-assay variations below <15% and a dilution recovery of approximately 100%.

### EXAMPLE 3

### Immunolocalization of the CIIM Fragment in Human OA Cartilage

Immunolocalization of the CIIM fragment in human OA cartilage was studied using the NB44-3C1 monoclonal antibody at different sites of the articular cartilage. Results are shown in FIG. 1 as follows:
(A) Negative control, from the upper zone to the subchondral bone, NB44-3C1 pre-incubated with immunogen (magnification 13.2×).
(B) Overview picture, NB44-3C1 staining from upper zone to subchondral bone (magnification 13.2×).
(C) The upper zone of the articular cartilage with zone surface irregularities (magnification 33×).
(D) The upper zone with surface erosion and fibrillation (magnification 33×).
(E) Mid zone with deep fissure damage (magnification 33×).
(F) Deep zone, calcified cartilage and subchondral bone (magnification 33×).

In FIG. 1B intense staining with NB44-3C1 (red colour—dark in monochrome) is observed in the upper zone (near cartilage surface) and lower sections (near the subchondral bone) of the tissue section. FIG. 1C demonstrates intense staining in upper zone at higher magnification. In FIG. 1D and E intense staining is observed in the upper zone, and in particular in areas of cartilage damage. Finally, FIG. 1F demonstrates intense staining with NB44-3C1 in the calcified cartilage proximal to the subchondral bone.

It was shown therefore that CIIM was immunolocalized to classical arthritis features in OA cartilage.

### EXAMPLE 4

### Measurement of Reactive Type II Collagen Fragments in Serum Samples Obtained from Patients with Rheumatoid Arthritis

Using the competition CIIM ELISA with monoclonal antibody NB44-3C1 described in Example 2, CIIM was measured in serum. Results are seen in FIG. 2. Serum samples were obtained from patients with rheumatoid arthritis before and after 4 weeks of treatment with anti-inflammatory therapy (solid line) or placebo (dashed line). Samples were stored at −20° C. until analysis for CIIM fragments by CIIM competition ELISA. At week 4, the difference between the placebo group and the treatment group was highly significant (p<0.05).

### EXAMPLE 5

### Correlation of Levels of Serum CIIM with OA Severity Scores

A total of 159 adult subjects aged 21 year or over from the greater Copenhagen area was invited to participate the CCBR study, as previously described. This population had varying degrees of OA symptoms, ranging from no symptoms to self-reported pain of 1 or 2 knees. Subjects with inflammatory arthritis, any contraindication for magnetic resonance imaging (MRI) examination, or previous knee joint replacement were excluded from the study. Digital X-rays of both knees were acquired simultaneously in the posterior-anterior position from every subject using SynaFlex from Synarc. The Kellgren-Lawrence (KL) index was determined for each lateral and medial tibio-femoral joints, with a score of 0 indicating little or no sign of knee OA and a maximum of 4 indicating progressive knee OA. Using the score of the knee worse of, subjects were divided into three subgroups: without OA (KL 0), mild OA (KL 1-2) and severe OA (3-4). Serum samples from 156 subjects were thawed from −80° C. storage and the level of CIIM measured. Serum samples from the other three subjects were either missing from the biobank or empty. The study was carried out in accordance with the Helsinki Declaration II and European Guidelines for Good Clinical Practice. The protocol was approved by the Danish National Committee on Biomedical Research Ethics (approval no. KA 2006-0054, Danish Ministry of Interior and Health). All participants signed approved information consent forms

CIIM was measured in all serum samples described above. There was no significant (p=0.323) difference in serum CIIM levels between women (0.778 ng/ml [CI95%; 0.693-0.907], n=75) and men (0.859 ng/ml [CI95%; 0.757-0.939], n=81). Subjects were subdivided into three groups depending on the degree of knee OA; without OA, mild OA and severe OA. The “No OA” group was significantly younger and had a significantly lower BMI than either of the OA groups. There was no significant difference in age and BMI between the mild and severe OA groups. Mean serum CIIM was significantly higher in individuals with mild OA (0.895 ng/ml, n=62) and severe OA (1.039 ng/ml, n=19) compared to individuals without OA (0.712 ng/ml, n=75) (FIG. 3). Although there was no significant difference between individuals with mild and severe OA, there was a trend towards increased levels of serum CIIM in those with severe OA.

### EXAMPLE 6

### CIIM Levels in Synovial Fluid

The CIIM ELISA was used to detect type II collagen fragments in synovial fluid. The mean level of CIIM measured in the synovial fluids from 51 OA patients was 0.850 ng/ml, with a range of 0.147 to 4.13 ng/ml.

### EXAMPLE 7

### Reactivity of Monoclonal Antibodies with Different Analytes

Monoclonal antibody 3C1 was compared to five other monoclonal antibodies raised against the same immunogen, i.e. KLH-C-GGGRDGAAG. All were reactive with the target sequence, i.e. PPGRDAAG, however, MAb 3C1 provided the best relative binding properties against analytes present in human body fluids. In particular, good reactivity was detected to analytes in human urine and human serum.

TABLE 1
Monoclonal antibody candidates

| Antigen | 3C1 | 6A11 | 4F7 | 2C9 | 5H3 | 2A2 |
|---|---|---|---|---|---|---|
| PPGRDGAAG | +++ | +++ | +++ | +++ | +++ | +++ |
| PPGRDGAAGV (elongated) | − | − | − | − | − | − |
| GFGLPGAAG (col I) | − | − | − | − | − | − |
| ETGERGAAG (col III) | − | − | − | − | − | − |
| KGATGPLGPK (nonsense) | − | − | − | − | − | + |
| uncleaved col II | − | − | − | − | − | − |
| MMP cleaved col II | +++ | +++ | +++ | +++ | +++ | +++ |
| HEX supernatant | +++ | +++ | ++ | +++ | − | +++ |
| BEX supernatant | +++ | +++ | ++ | ++ | + | ++ |
| Sensitivity in human | | | | | | |
| bodyfluids: | | | | | | |
| Human urine | ++ | + | − | − | − | − |
| Human serum | +++ | ++ | ++ | − | − | − |
| Synovial fluid | ++ | ++ | ++ | − | − | − |

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

1. A method of assay for Type II collagen fragments in serum, plasma, or synovial fluid comprising obtaining a quantitative measure of the concentration of protein fragments in a serum, plasma, or synovial fluid sample that are reactive with an antibody, or immunoreactive antibody fragment, having specific reactivity with a C-terminal epitope present in the amino acid sequence GPPGRDGAAG and lacking specific reactivity with an amino acid sequence comprising the amino acid sequence GPPGRDGAAGV.

2. The method as claimed in claim 1, wherein no subset of protein fragments that are reactive with said antibody, or immunoreactive antibody fragment, having specific reactivity with a C-terminal epitope present in the amino acid sequence GPPGRDGAAG and lacking specific reactivity with an amino acid sequence comprising the amino acid sequence GPPGRDGAAGV are excluded from the measurement.

3. The method as claimed in claim 1, comprising contacting protein fragments present in a said serum sample with a said antibody or immunoreactive antibody fragment and measuring the amount of binding of said fragments thereto.

4. The method as claimed in claim 3, wherein said antibody or immunoreactive antibody fragment is contacted with both said protein fragments and a competition agent for which said antibody or immunoreactive antibody fragment has specific binding affinity.

5. The method as claimed in claim 1 wherein the antibody is NB44-3C1 as deposited in HPA Culture Collection Logistics Office, Health Protection Agency Culture Collections, Centre For Emergency Preparedness and Response, Porton Down, Salisbury Wiltshire, SP4 0JG, UK, with Accession Number 10091402.

6. Antibody NB44-3C1 as produced by the cell line deposited in HPA Culture Collection Logistics Office, Health Protection Agency Culture Collections, Centre For Emergency Preparedness and Response, Porton Down, Salisbury Wiltshire, SP4 0JG, UK, with Accession Number 10091402.

7. The antibody as claimed in claim 6, labelled with a detectable label.

8. The antibody as claimed in claim 7, wherein said label is a peroxidase enzyme label.

9. A test kit for conducting an immunoassay comprising antibody NB44-3C1 as claimed in claim 6, together with one or more of, standards for generating a calibration curve, a peptide immunologically reactive with said antibody, a chromogenic substrate capable of participating in a colour forming reaction, a stopping solution for a colour forming reaction, or a multi-well assay plate.


SEQUENCE LISTING (22 sequences)

SEQ ID NO: 1 - protein, 16 aa, Homo sapiens
  SOURCE 1..16: /mol_type=“protein” /organism=“Homo sapiens”
GSPGADGPPGRDGAAG

SEQ ID NO: 2 - protein, 27 aa, Homo sapiens
  SOURCE 1..27: /mol_type=“protein” /organism=“Homo sapiens”
LTGPAGEPGREGSPGADGPPGRDGAAG

SEQ ID NO: 3 - protein, 6 aa, Homo sapiens
  SOURCE 1..6: /mol_type=“protein” /organism=“Homo sapiens”
LTGPAG

SEQ ID NO: 4 - protein, 6 aa, Homo sapiens
  SOURCE 1..6: /mol_type=“protein” /organism=“Homo sapiens”
RDGAAG

SEQ ID NO: 5 - protein, 1060 aa, Homo sapiens
  SOURCE 1..1060: /mol_type=“protein” /organism=“Homo sapiens”
QMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRA

SEQ ID NO: 6 - protein, 9 aa, Homo sapiens
  SOURCE 1..9: /mol_type=“protein” /organism=“Homo sapiens”
PPGRDGAAG

SEQ ID NO: 7 - protein, 10 aa, Homo sapiens
  SOURCE 1..10: /mol_type=“protein” /organism=“Homo sapiens”
PPGRDGAAGV

SEQ ID NO: 8 - protein, 9 aa, Homo sapiens
  SOURCE 1..9: /mol_type=“protein” /organism=“Homo sapiens”
ETGERGAAG

SEQ ID NO: 9 - protein, 9 aa, Homo sapiens
  SOURCE 1..9: /mol_type=“protein” /organism=“Homo sapiens”
GFGLPGAAG

SEQ ID NO: 10 - protein, 9 aa, Homo sapiens
  SOURCE 1..9: /mol_type=“protein” /organism=“Homo sapiens”
DQGVPGEAG

SEQ ID NO: 11 - protein, 9 aa, Homo sapiens
  SOURCE 1..9: /mol_type=“protein” /organism=“Homo sapiens”
REGSPGASG

SEQ ID NO: 12 - protein, 9 aa, Homo sapiens
  SOURCE 1..9: /mol_type=“protein” /organism=“Homo sapiens”
LAGPKGANG

SEQ ID NO: 13 - protein, 9 aa, Homo sapiens
  SOURCE 1..9: /mol_type=“protein” /organism=“Homo sapiens”
DDGEAGKPG

SEQ ID NO: 14 - protein, 9 aa, Homo sapiens
  SOURCE 1..9: /mol_type=“protein” /organism=“Homo sapiens”
ATGPLGPKG

SEQ ID NO: 15 - protein, 9 aa, Homo sapiens
  SOURCE 1..9: /mol_type=“protein” /organism=“Homo sapiens”
LTGPAGEPG

SEQ ID NO: 16 - protein, 9 aa, Homo sapiens
  SOURCE 1..9: /mol_type=“protein” /organism=“Homo sapiens”
GADGPPGRD

SEQ ID NO: 17 - protein, 10 aa, Homo sapiens
  SOURCE 1..10: /mol_type=“protein” /organism=“Homo sapiens”
GPPGRDGAAG

SEQ ID NO: 18 - protein, 11 aa, Homo sapiens
  SOURCE 1..11: /mol_type=“protein” /organism=“Homo sapiens”
GPPGRDGAAGV

SEQ ID NO: 19 - protein, 10 aa, Homo sapiens
  SOURCE 1..10: /mol_type=“protein” /organism=“Homo sapiens”
CGGGRDGAAG

SEQ ID NO: 20 - protein, 10 aa, Homo sapiens
  SOURCE 1..10: /mol_type=“protein” /organism=“Homo sapiens”
KPPGRDGAAG

SEQ ID NO: 21 - protein, 11 aa, Homo sapiens
  SOURCE 1..11: /mol_type=“protein” /organism=“Homo sapiens”
KPPGRDGAAGV

SEQ ID NO: 22 - protein, 10 aa, artificial sequence
  SOURCE 1..10: /mol_type=“protein” /note=“nonsense sequence” /organism=“artificial sequence”
KGATGPLGPK